(19) United States
(12) Patent Application Publication
(10) Pub. No.: US 2026/0365964 A1
(43) Pub. Date: Nov. 19, 2026

(54) AUTOMATED CELL CULTURE HARVESTING SYSTEM

(71) Applicant: CellFabs Inc., Tainan City (TW)

(72) Inventors: Yun-Shu Chiou, Tainan City (TW); Shang-Chih Lin, Tainan City (TW)

(73) Assignee: CellFabs Inc., Tainan City (TW)

(21) Appl. No.: 19/206,595

(22) Filed: May 13, 2025

(51) Int. Cl. C12M 1/36; B25J 9/00; C12M 1/00; C12M 1/12; C12M 1/26; C12M 3/00

(52) U.S. Cl. CPC C12M 41/48; B25J 9/0087; C12M 23/42; C12M 33/07; C12M 37/00; C12M 41/14

(57) ABSTRACT

A harvest system for an automated cell culture room, wherein the culture bottle transfer mechanism transports the cell culture flask to a designated position, and the first robotic arm grips the pipette tube and inserts it into the cell culture flask to extract the culture medium, which is then transferred into the harvest bottle for storage. The second robotic arm then grips the harvest bottle and places it into the centrifuge for cell separation. After centrifugation, the harvest flask supply mechanism transports the harvest bottle, during which the valve body can be actuated to form a sealed state, allowing the harvest tube to be stored independently. This invention utilizes track-based delivery and the coordinated operation of multiple mechanisms to achieve full-process automation of cell culture, extraction, harvesting, centrifugation, and storage. It not only enhances operational efficiency but also reduces the risk of contamination, making it suitable for the high-efficiency production needs of large-scale automated cell culture facilities.

### BACKGROUND OF THE DISCLOSURE

### 1. Field of the Disclosure

The present invention relates to a cell culture and harvesting technology, and more particularly to an automated harvesting system suitable for use in large-scale cell culture facilities, which is capable of automatically performing the extraction of cell culture medium, centrifugal separation of cells, and harvesting of cells.

### 2. Description of the Prior Art

In current practices, cell culture and harvesting operations primarily rely on manual handling of cell culture flasks, extraction of culture media, centrifugation, and harvesting processes. While manual operations offer flexibility to adapt to various culture conditions, the increasing scale of cell cultivation and the growing demands for product quality and biosafety have exposed numerous limitations and risks in traditional manual workflows. Particularly in high-frequency or large-scale production scenarios, manual handling is prone to contamination, cross-infection, or inconsistent procedures due to human error, potentially compromising cell viability. Moreover, the need for specialized personnel to maintain high-standard operating environments results in persistently high labor costs, further constraining the scalability and mass production capacity of cell preparation facilities.

Therefore, some automated systems have attempted to incorporate robotic arms for partial assistance, such as handling culture flasks and transferring liquids. However, current technologies often focus on isolated operational steps and lack an integrated automation design for the entire cell harvesting process. For instance, during critical procedures such as culture medium extraction, transfer to the harvest bottle, centrifugation, and cell preservation, traditional culture flask and harvest bottle designs do not accommodate robotic manipulation needs. Their dimensions and opening configurations are not conducive to gripping, positioning, or sealing, thereby still requiring manual intervention for tasks such as opening caps, tube insertion, transportation, and centrifugation. This semi-automated approach not only fails to meet the comprehensive demands for high efficiency, full automation, and closed sterile environments, but also increases the risk of contamination and human error, ultimately limiting the potential for clinical application and large-scale production of cell-based products.

In existing cell culture systems, the supply and collection of auxiliary tools such as pipette tubes and harvest bottles are still predominantly carried out manually. This lack of standardized planning and automated management increases operational complexity and the risk of equipment contamination. Particularly during high-volume operations in sterile environments, frequent human intervention significantly raises the difficulty of maintaining biosafety control, further affecting the consistency and reproducibility of the cell processing workflow.

In view of the foregoing, the inventor, drawing upon years of experience in the development, manufacturing, and design of related products, has conducted detailed design and careful evaluation to ultimately achieve the present invention, which is both practical and effective.

### SUMMARY OF THE DISCLOSURE

The technical problem intended to be solved by the present invention is to address the aforementioned deficiencies in the prior art by providing an automated harvesting system for cell culture rooms.

A culture bottle transfer mechanism includes a main rail and a plurality of moving platforms slidably disposed on the main rail, each moving platform being configured to place and transport a cell culture flask; a first robotic arm includes a base connected to an arm top section capable of three-dimensional movement, wherein the arm top section is provided with a lower gripper arm and an upper gripper arm; a pipette tube transport mechanism includes a transport rail and a plurality of transport carts slidably disposed on the transport rail, each transport cart being configured to place and transport at least one pipette tube. The pipette tube includes a tube body, a plunger rod, and a tubing, with one end of the tube body connected to the tubing and the other end formed with an expanded opening through which the plunger rod is inserted. The first robotic arm uses the lower gripper arm to grip the tube body and the upper gripper arm to grip the plunger rod, allowing the tubing to be inserted into the cell culture flask, and then moves the upper gripper arm to extract liquid by pulling the plunger rod; a harvest flask supply mechanism includes a supply rail and a plurality of supply carts slidably disposed on the supply rail, each supply cart being configured to place and transport at least one harvest bottle. The harvest bottle includes a bottle body, a lid assembly, and a harvest tube, where the lid assembly is fixed to cover one open end of the bottle body, and the harvest tube is fixed to the other open end of the bottle body. The first robotic arm controls the pipette tube's tubing to insert into the bottle body and pushes the plunger rod to transfer the cultured cell liquid into the bottle body; a second robotic arm includes a mounting base connected to a gripper arm capable of three-dimensional movement, and the gripper arm is provided with two gripper jaws; a centrifuge includes a motor driving a support frame, with at least one rotary tube suspended from the support frame. The second robotic arm uses the gripper jaws to grip the harvest bottle containing cultured cells and places the harvest bottle into the rotary tube. The motor drives the support frame to rotate, and the rotary tube is swung outward to sediment the cells into the harvest tube through centrifugal force.

The arm top section is connected to a first linear guide rail, and the first robotic arm controls the first linear guide rail to be normally oriented vertically to the ground. A first slider block and a second slider block are slidably disposed on the first linear guide rail, wherein the lower gripper arm is coupled to the first slider block, and the upper gripper arm is coupled to the second slider block.

The harvest tube is formed with a valve chamber, and a valve body is disposed within the valve chamber. The valve body is penetrated by a flow channel, and by rotating the valve body, the flow channel can be selectively opened or closed to control communication with the interior of the harvest tube.

The outer surface of the bottle body is formed with a first collar, and the bottle body is secured to the rotary tube of the centrifuge via the first collar. The outer surface of the bottle body is also formed with a second collar, and the space between the first collar and the second collar is configured to allow the gripper jaw of the second robotic arm to grasp and transfer the bottle body.

The lid assembly is provided with a window, and a coupling section is formed adjacent to the window. A cover plate is pivotally mounted to the coupling section via a latch pin, and a torsion spring is fitted around the latch pin. The torsion spring elastically presses against both the cover plate and the coupling section, thereby maintaining the cover plate in a normally closed position over the window.

The transport cart is provided with at least one insertion slot, against which the pipette tube is positioned so that the tube body is naturally oriented downward. Additionally, the transport cart extends downward to include a liquid collection tray, which is located directly beneath the tube body.

The centrifuge comprises a housing that contains the motor, support frame, and rotary tube. An operation window is formed on the top of the housing, through which the second robotic arm places and retrieves the harvest bottle. Additionally, multiple UV sterilization lamps are installed inside the housing to perform ultraviolet sterilization when the centrifuge is not in use.

The gripper arm is connected to a second linear guide rail, and the second robotic arm controls the second linear guide rail to remain normally vertical to the ground. A third slider block is slidably disposed on the second linear guide rail, and the gripper jaw is coupled to the third slider block.

The harvest flask supply mechanism further includes a harvest rail, along which a plurality of harvest carts are slidably disposed. After the harvest bottle completes cell separation within the centrifuge, the second robotic arm retrieves the harvest bottle and places the centrifuged harvest bottle onto the corresponding harvest cart.

The pipette tube transport mechanism further includes a recovery track, along which a plurality of recovery carts are slidably disposed. After use, the pipette tube is placed onto the recovery cart by the first robotic arm.

The primary objective of the present invention is that the culture bottle transfer mechanism is provided with the main rail and the moving platform. When the cell culture status meets the harvesting conditions, the moving platform automatically transfers the cell culture flask to the designated harvesting location via the main rail. The first robotic arm is equipped with independently movable lower gripper arm and upper gripper arm, allowing the tube body and the plunger rod of the pipette tube to be precisely gripped and operated separately. The tube body is used to extract cell culture fluid from the cell culture flask. Additionally, the centrifuge concentrates and deposits the cells into the harvest tube, while the second robotic arm accurately places and retrieves the harvest bottle using the gripper jaw. This design significantly improves the accuracy and stability of the liquid extraction, injection, and centrifugation processes, effectively avoiding manual sampling errors and contamination risks, enhancing screening efficiency, and increasing the automation level of the culture facility to ensure consistency in cell quality and production flow.

The second primary objective of the present invention lies in that the bottle body of the harvest bottle is externally formed with the first collar and the second collar. The first collar is used to stably engage with the rotary tube of the centrifuge, while the second collar provides a gripping section for the gripper jaw of the second robotic arm. Through this dual-collar design, the harvest bottle can be securely retained during placement and centrifugation, and after the centrifugation process is completed, it can be gripped and transferred quickly and safely. This significantly enhances the safety and efficiency of the cell separation and harvesting operations and reduces the risk of cell loss caused by the harvest bottle slipping or being damaged during operation.

The third primary objective of the present invention lies in that the lid assembly of the harvest bottle is provided with the cover plate, which is supported by the latch pin and the torsion spring to shield the window formed on the lid assembly. The cover plate is designed to automatically return to a closed state, thereby preventing external contaminants from entering the harvest bottle during operation intervals. This self-closing structural design effectively ensures a sterile environment throughout the cell harvesting process, while eliminating the need for complex lid assembly opening and closing operations. As a result, it enhances the automation level and operational convenience of the workflow, meeting the stringent cleanliness standards required for cell processing.

Other objects, advantages, and novel features of the present invention will become more apparent from the following detailed description and the accompanying drawings.

### BRIEF DESCRIPTION OF THE DRAWINGS

The present invention will be best understood by referring to the following detailed description of one illustrative embodiment in conjunction with the accompanying drawings, in which

FIG. 1 is a perspective view of the present invention;

FIG. 2 is a top view of the present invention;

FIG. 3 is another perspective view of the present invention from a different angle;

FIG. 4 is an exploded perspective view of the cell culture flask of the present invention;

FIG. 5 is an exploded perspective view of the pipette tube transport mechanism of the present invention;

FIG. 6 is a schematic view illustrating the extraction of culture medium using the pipette tube (1) of the present invention;

FIG. 7 is a schematic view illustrating the extraction of culture medium using the pipette tube (2) of the present invention;

FIG. 8 is a schematic view illustrating the extraction of culture medium using the pipette tube (3) of the present invention;

FIG. 9 is a schematic view showing the filling of culture medium into the harvest bottle of the present invention;

FIG. 10 is a schematic view showing the placement of the harvest bottle into the centrifuge of the present invention;

FIG. 11 is an exploded perspective view of the harvest bottle of the present invention;

FIG. 12 is an internal perspective view of the centrifuge applied in the present invention;

FIG. 13 is a schematic view of the centrifugation operation of the present invention;

FIG. 14 is a schematic view of the harvest bottle used to collect sedimented cells in the present invention;

FIG. 15 is a schematic view of the harvest bottle with the valve body closed according to the present invention; and

FIG. 16 is a schematic view of separating the bottle body from the harvest tube according to the present invention.

### DETAILED DESCRIPTION

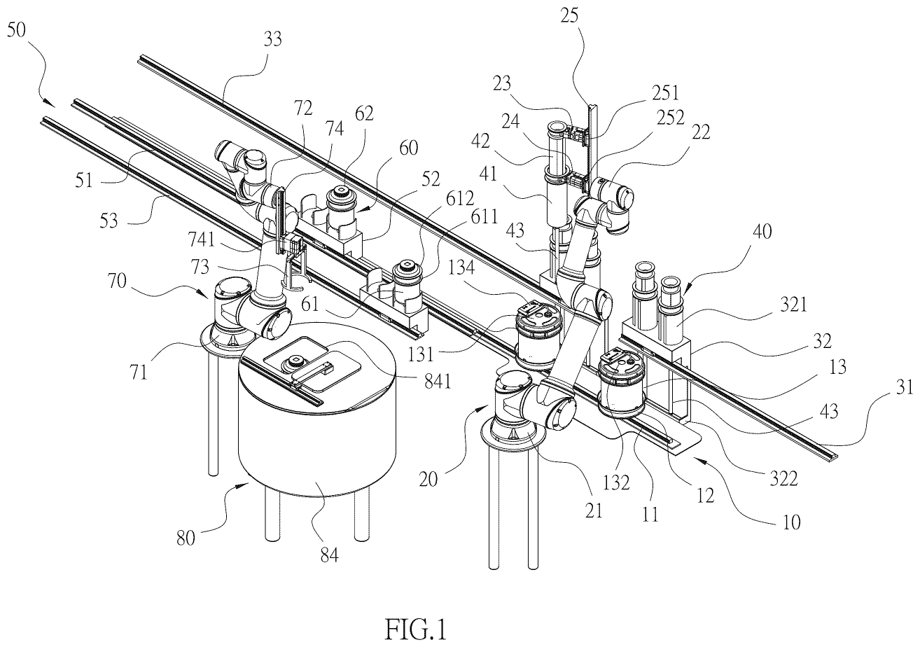

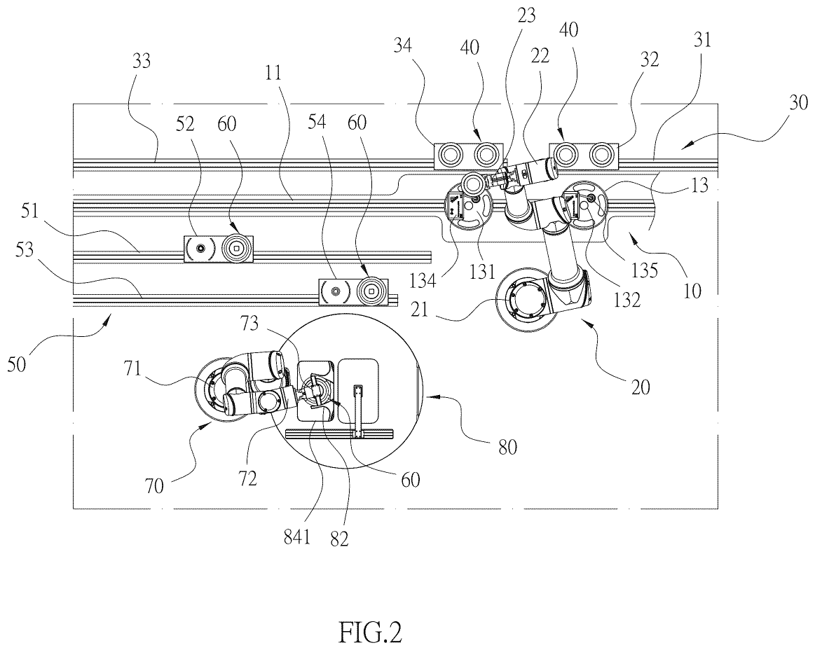

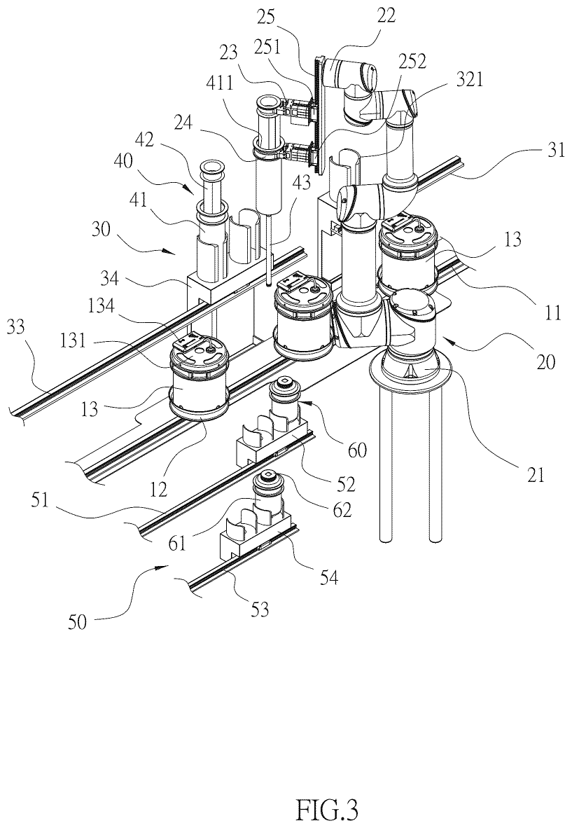

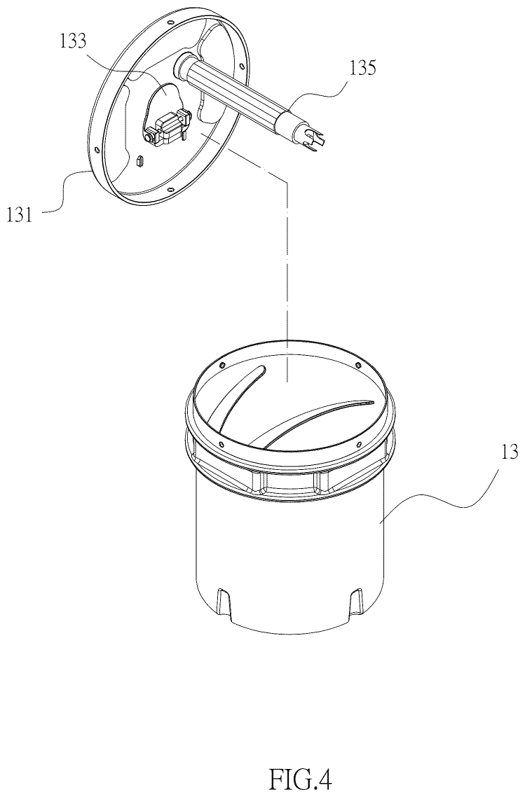

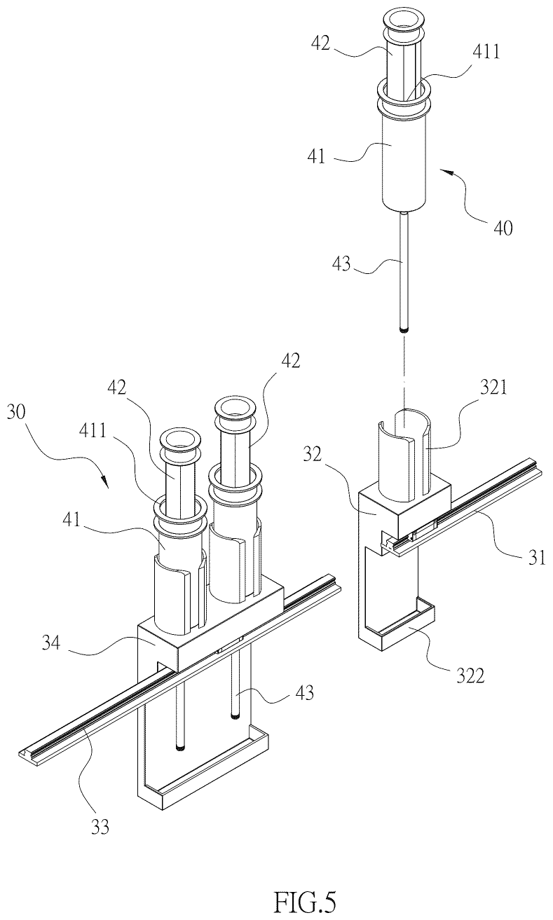

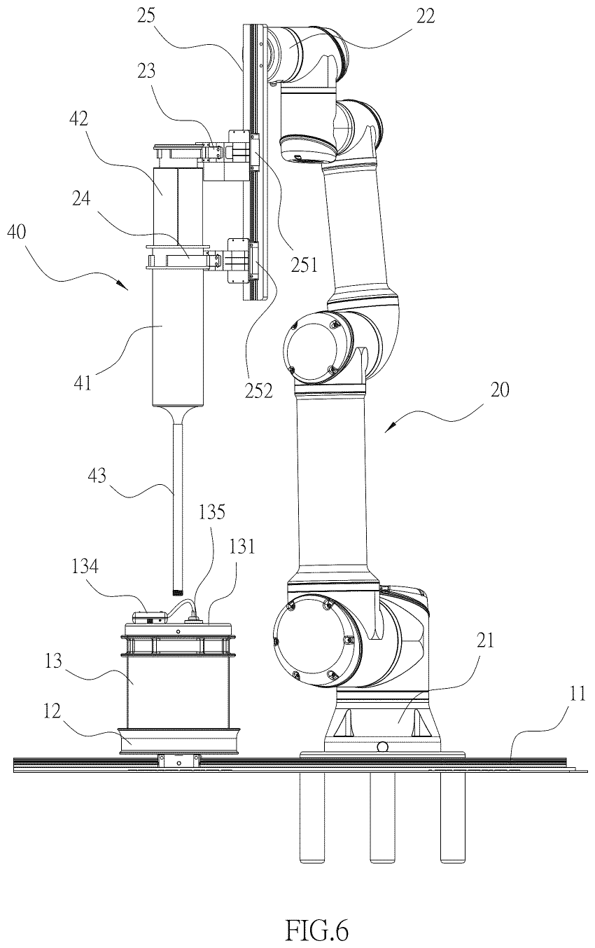

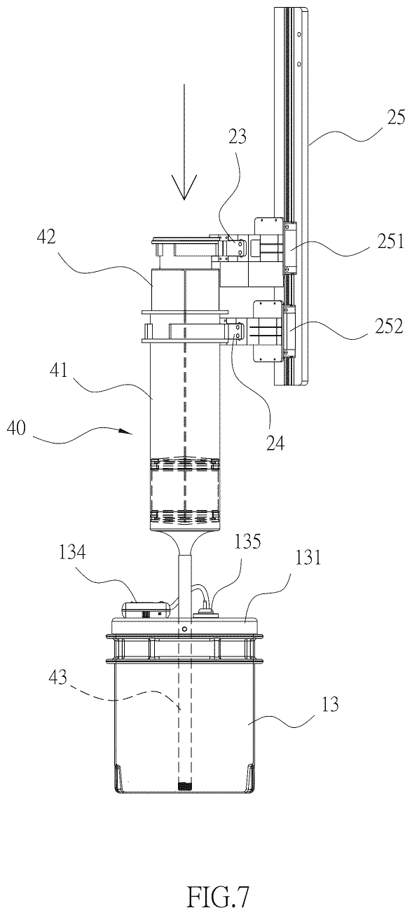

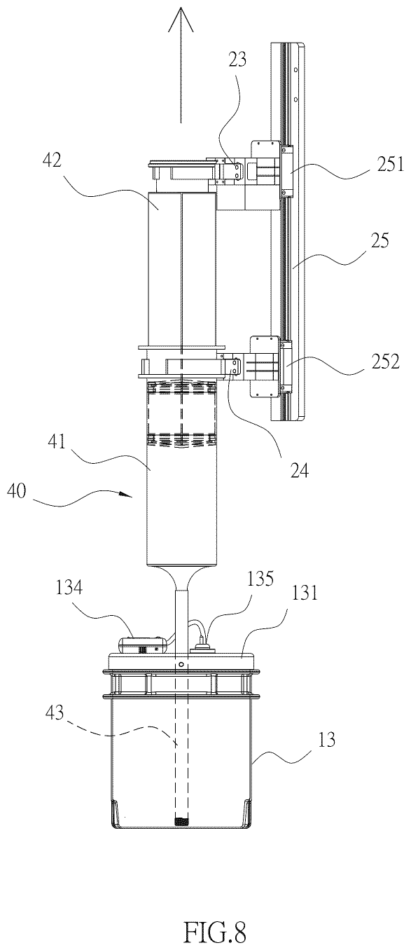

Referring first to FIGS. 1 through 9, an automated harvesting system for a cell culture room includes: a culture bottle transfer mechanism 10, a first robotic arm 20, a pipette tube transport mechanism 30, at least one pipette tube 40, a harvest flask supply mechanism 50, at least one harvest bottle 60, a second robotic arm 70, and a centrifuge 80. Through the coordinated operation of the aforementioned mechanisms, an automated workflow is achieved for extracting culture medium, separating cells via centrifugation, and collecting the cells. Furthermore, all mechanisms are interconnected through a signal control module to enable a fully automated process and reduce the risk of contamination caused by manual intervention. Among them:

A culture bottle transfer mechanism 10 includes a main rail 11 and a plurality of moving platforms 12 slidably disposed on the main rail 11. Each moving platform 12 is configured to place and transport a cell culture flask 13, and is capable of precisely moving along a designated direction to a predetermined location, offering high positioning accuracy and transport efficiency. The cell culture flask 13 is sealed with a flask cap 131, which has a centrally located cap opening 132. A door panel 133 is pivotally connected to the cap opening 132 and is elastically positioned to normally close the opening, while also being easily opened upon application of external force. Additionally, a pH sensor 134 is fixed on the outside of the flask cap 131, with a sensing probe 135 inserted into the interior of the cell culture flask 13. This sensing probe 135 continuously monitors pH variations of the culture medium and transmits data to a central control system for analysis. When significant pH changes are detected, the corresponding cell culture flask 13 is removed for inspection. Based on the inspection result, flasks deemed ready for harvest are placed on the moving platform 12 and transported via the main rail 11 to a designated location within the harvest area.

A first robotic arm 20 is connected to a base 21 and includes an arm top section 22 capable of three-dimensional spatial movement. The arm top section 22 is formed with a lower gripper arm 23 and an upper gripper arm 24. The arm top section 22 is further connected to a first linear guide rail 25, which is vertically oriented under the control of the first robotic arm 20. This first linear guide rail 25 provides precise linear displacement in the vertical direction, enabling the first robotic arm 20 to perform accurate positioning for operations requiring different height levels. A first slider block 251 and a second slider block 252 are slidably mounted on the first linear guide rail 25. The lower gripper arm 23 is coupled to the first slider block 251, while the upper gripper arm 24 is coupled to the second slider block 252. By controlling the relative movement between the first slider block 251 and the second slider block 252, the relative motion between the lower gripper arm 23 and the upper gripper arm 24 can be precisely regulated. This configuration allows for more accurate and reliable gripping and release of the pipette tube 40, thereby enhancing operational stability and efficiency.

A pipette tube transport mechanism 30 includes a transport rail 31, and multiple transport carts 32 slidably mounted on the transport rail 31. Each transport cart 32 is configured to carry and deliver at least one pipette tube 40. The pipette tube 40 comprises a tube body 41, a plunger rod 42, and a tubing 43. One end of the tube body 41 is in fluid communication with the tubing 43, while the opposite end of the tube body 41 forms an expanded opening 411, through which the plunger rod 42 is inserted into the tube body 41. This allows the plunger rod 42 to be pushed or pulled along the interior of the tube body 41 to perform liquid suction or dispensing operations. The first robotic arm 20 grips the tube body 41 with the lower gripper arm 23 and the plunger rod 42 with the upper gripper arm 24, inserting the tubing 43 through the cap opening 132 into the cell culture flask 13. The upper gripper arm 24 then pulls the plunger rod 42 to extract the culture medium, enabling precise control of the suction rate and volume, thereby preventing splashing or contamination. The transport cart 32 is provided with at least one insertion slot 321, against which the pipette tube 40 is placed so that the tubing 43 naturally hangs downward. This orientation utilizes gravity to assist in concentrating residual liquid at the bottom of the tubing 43 for easier disposal. Additionally, the transport cart 32 extends downward to include a liquid collection tray 322 located directly beneath the tubing 43, thereby preventing any residual liquid from dripping onto the ground or other equipment surfaces, which helps maintain cleanliness and simplifies maintenance. Furthermore, the pipette tube transport mechanism 30 includes a recovery track 33 with multiple recovery carts 34 slidably mounted thereon. Used pipette tubes 40 are placed into the recovery carts 34 by the first robotic arm 20, allowing for automated retrieval and contributing to enhanced automation and aseptic handling throughout the system.

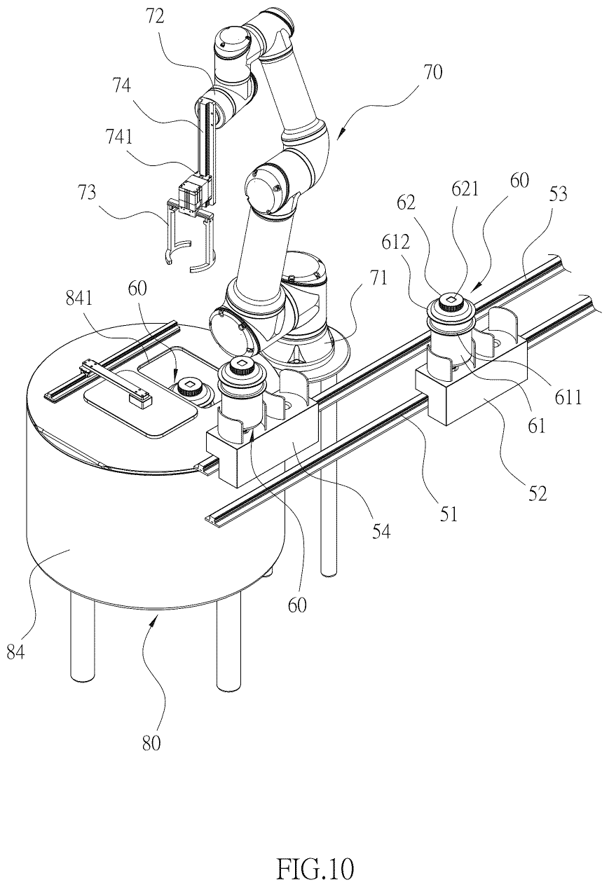

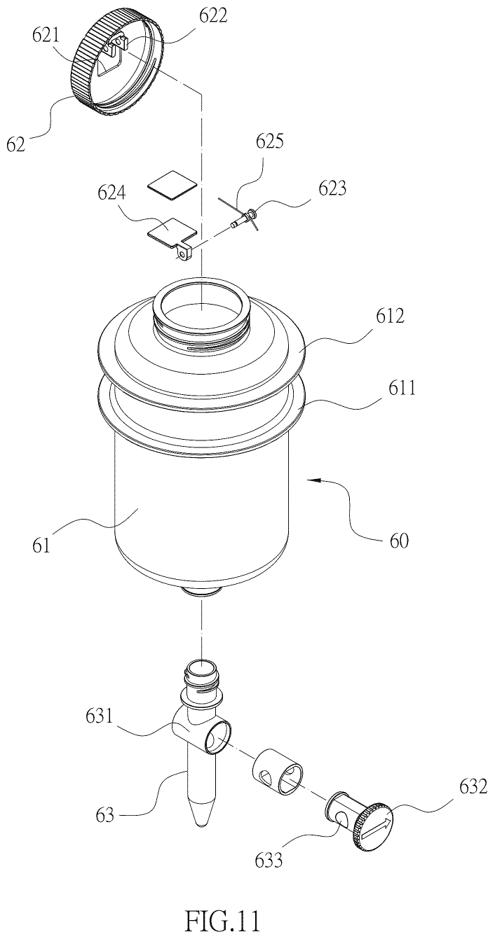

Referring also to FIG. 10 and FIG. 11, a harvest flask supply mechanism 50 includes a supply rail 51, and multiple supply carts 52 slidably mounted on the supply rail 51. Each supply cart 52 is configured to store and transport at least one harvest bottle 60. The harvest bottle 60 comprises a bottle body 61, a lid assembly 62, and a harvest tube 63. The lid assembly 62 is fixed to seal one opening of the bottle body 61, and the harvest tube 63 is fixed to the opposite end of the bottle body 61 in fluid communication, thereby forming a sealed structure that facilitates subsequent collection and preservation of the cell culture medium. The first robotic arm 20 controls the pipette tube 40 by inserting its tubing 43 into the bottle body 61 and pushing the plunger rod 42 to transfer the culture medium into the bottle body 61. The linear motion of the plunger rod 42 allows control over the inflow speed and the fluid level inside the bottle. The harvest tube 63 includes a valve chamber 631, and a valve body 632 is installed inside the valve chamber 631. The valve body 632 is penetrated by a flow channel 633. Rotating the valve body 632 enables the flow channel 633 to be opened or closed, thereby meeting the operational needs of cell isolation and subsequent processing, allowing the harvest tube 63 to be separated and stored independently. The lid assembly 62 has a window 621, and a coupling section 622 is raised near the window 621. A cover plate 624 is pivotally mounted to the coupling section 622 via a latch pin 623, and a torsion spring 625 is also mounted on the latch pin 623. The torsion spring 625 elastically presses between the cover plate 624 and the coupling section 622, enabling the cover plate 624 to remain normally closed over the window 621 due to the restoring force of the spring. The harvest flask supply mechanism 50 further includes a harvest rail 53 and multiple harvest carts 54 slidably disposed along the harvest rail 53. After cells in the harvest bottle 60 have been separated in the centrifuge 80, the second robotic arm 70 retrieves the harvest bottle 60 and places the completed bottle onto the harvest cart 54. The modular configuration of the harvest cart 54 allows for flexible categorization and management of harvest bottles 60 according to different batch processes.

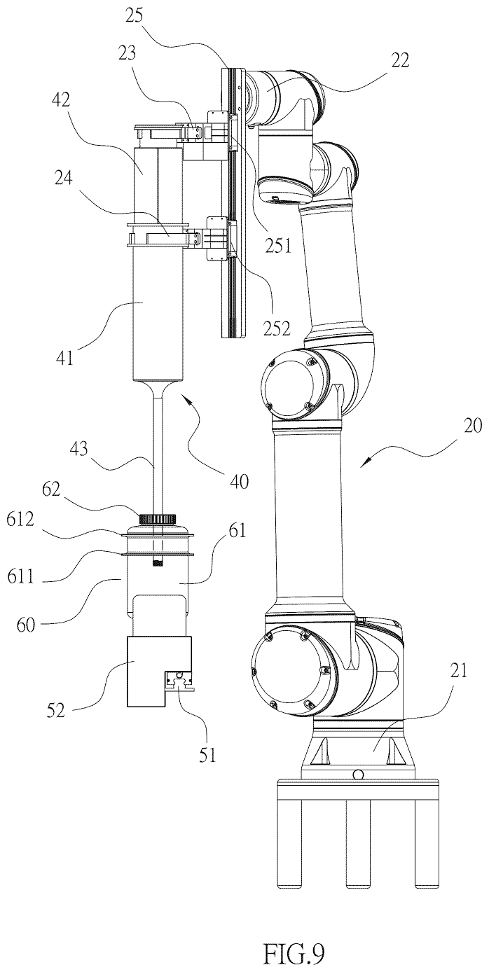

As shown in FIG. 9, a second robotic arm 70 includes a mounting base 71 connected to a gripper arm 72 that is capable of three-dimensional spatial movement. The gripper arm 72 is equipped with two gripper jaws 73, which operate synchronously to grasp the harvest bottle 60. The gripper arm 72 is further connected to a second linear guide rail 74, and the second robotic arm 70 controls the second linear guide rail 74 to maintain a vertical orientation. The linear motion provided by the second linear guide rail 74 enables vertical displacement of the gripper jaws 73, enhancing the flexibility of operation at various height positions. A third slider block 741 is slidably mounted on the second linear guide rail 74, and the gripper jaws 73 are attached to the third slider block 741. This allows the gripper jaws 73 to move up and down in conjunction with the third slider block 741, enabling precise grasping and placement operations. Such a configuration significantly improves the level of automation within the cell harvesting system.

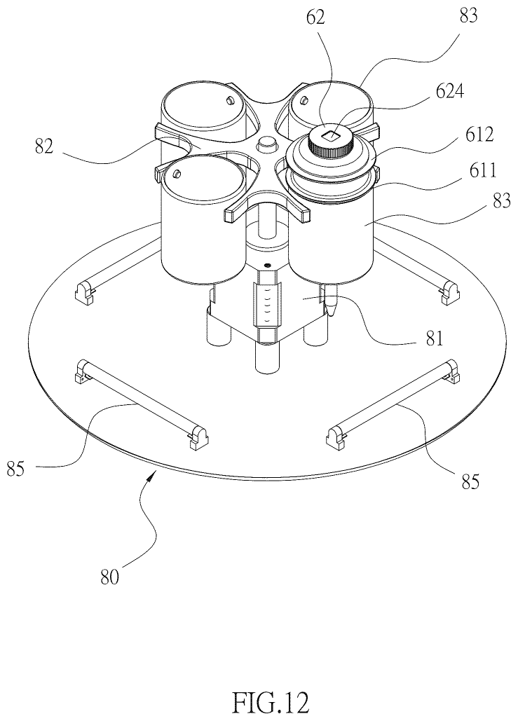

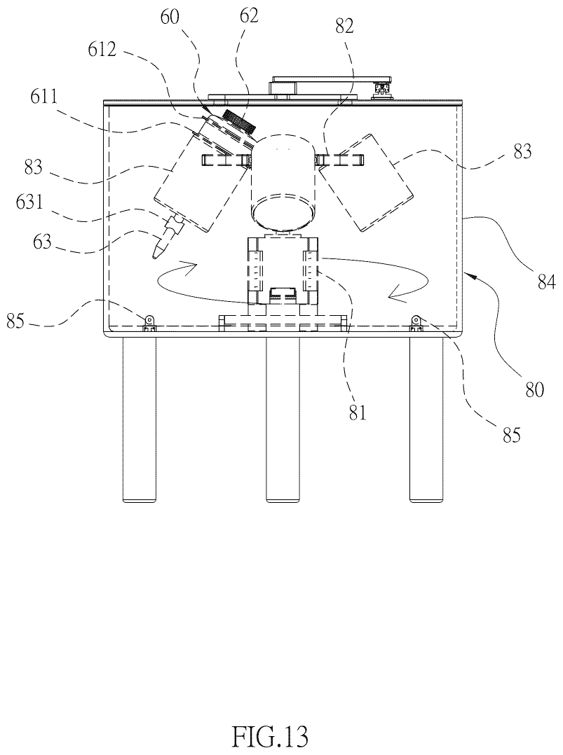

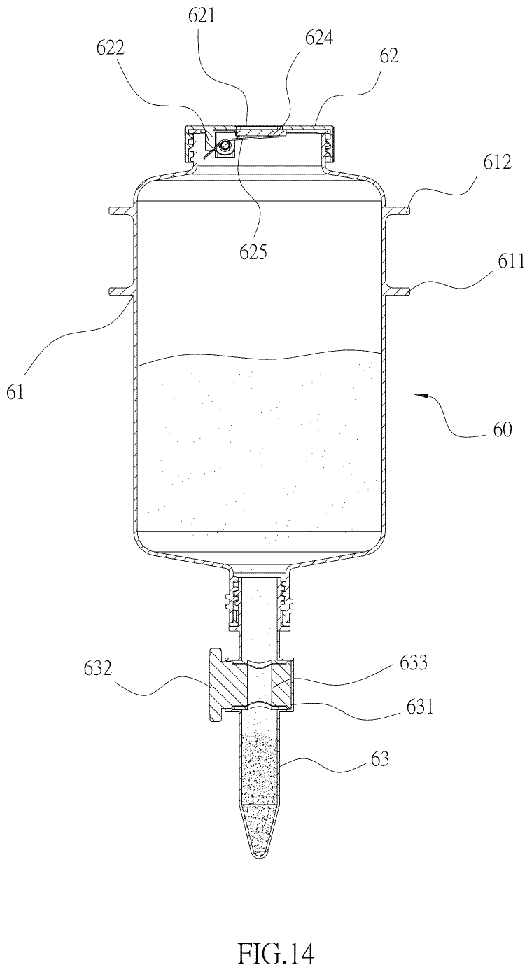

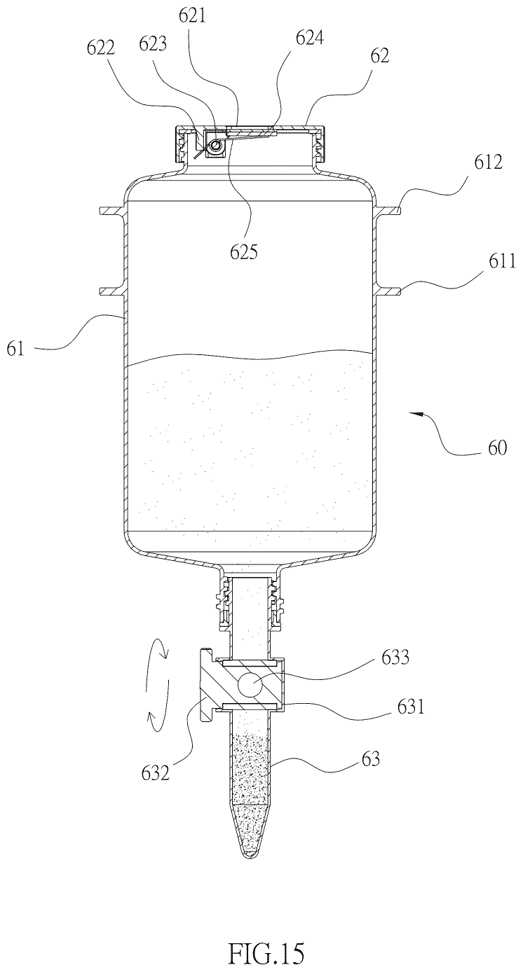

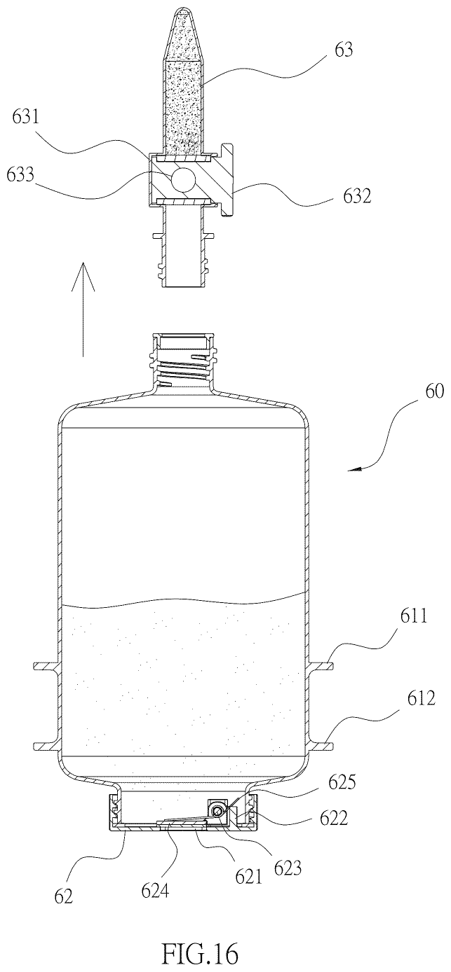

Referring also to FIGS. 9 through 16, a centrifuge 80 includes a motor 81 that drives a support frame 82. The support frame 82 is suspended with at least one rotary tube 83. The support frame 82 is driven by the motor 81 to rotate at high speed, generating the necessary centrifugal force for cell separation. The second robotic arm 70 uses the gripper jaws 73 to grasp a harvest bottle 60 containing cultured cell liquid and accurately positions it into the rotary tube 83. The placement operation ensures correct positioning of the harvest bottle 60 within the rotary tube 83, preventing displacement or damage during operation. The motor 81 rotates the support frame 82 and causes the rotary tube 83 to swing outward, using centrifugal force to deposit cells into the harvest tube 63. This allows cells to rapidly concentrate at the bottom of the harvest tube 63 in a short period, thereby improving collection efficiency and maintaining cell viability. Furthermore, the outer surface of the bottle body 61 is formed with a first collar 611, which secures the harvest bottle 60 in the rotary tube 83 of the centrifuge 80. The first collar 611 provides alignment and support functions, ensuring stability during centrifugation. Additionally, a second collar 612 is formed on the outer surface of the bottle body 61. The space between the first collar 611 and the second collar 612 is configured for the gripper jaws 73 of the second robotic arm 70 to grasp and move the harvest bottle 60. This design enables the second robotic arm 70 to grip or release the harvest bottle 60 quickly and securely, enhancing operational fluidity. The centrifuge 80 further includes a housing 84 that accommodates the motor 81, support frame 82, and rotary tube 83. An operation window 841 is formed at the top of the housing 84, allowing the second robotic arm 70 to place and retrieve the harvest bottle 60. The operation window 841 effectively maintains cleanliness within the centrifuge 80 and streamlines the movement path of the second robotic arm 70. Moreover, multiple UV sterilization lamps 85 are installed inside the housing 84. These lamps perform UV sterilization when the centrifuge 80 is not in use, helping maintain a sterile environment, reduce the risk of cell contamination, and enhance the reliability of the overall system.

The above description represents merely a preferred embodiment of the present invention and should not be construed as limiting the scope of implementation of the invention. Any equivalent changes or modifications made in accordance with the claims of the present invention shall fall within the scope of the patent protection of the present invention.

What is claimed is:

1. A harvest system for an automated cell culture room, comprising:
a culture bottle transfer mechanism, which comprises a main rail and a plurality of moving platforms slidably disposed on the main rail, each moving platform being used to place and transport a cell culture flask;
a first robotic arm, which connects a base to an arm top section capable of three-dimensional movement, and the arm top section is formed with a lower gripper arm and an upper gripper arm;
a pipette tube transport mechanism, which comprises a transport rail and a plurality of transport carts slidably disposed on the transport rail, each transport cart being used to place and transport at least one pipette tube, the pipette tube comprising a tube body, a plunger rod, and a tube body, one end of the tube body being in communication with and connected to the tube body, and the other end of the tube body being formed with an expanded opening allowing the plunger rod to pass through the expanded opening into the tube body; the first robotic arm grips the tube body with the lower gripper arm, and grips the plunger rod with the upper gripper arm, allowing the tube body to be inserted into the cell culture flask, and moves the upper gripper arm to pull the plunger rod to perform liquid extraction;
a harvest flask supply mechanism, which comprises a supply rail and a plurality of supply carts slidably disposed on the supply rail, each supply cart being used to place and transport at least one harvest bottle, the harvest bottle comprising a bottle body, a lid assembly, and a harvest tube, the lid assembly covering and fixed to one end opening of the bottle body, and the harvest tube being in communication with and fixed to the other end opening of the bottle body; the first robotic arm controls the tube body of the pipette tube to be inserted into the bottle body and pushes the plunger rod to transfer the cell culture liquid into the bottle body;
a second robotic arm, which connects a mounting base to a gripper arm capable of three-dimensional movement, and the gripper arm is formed with two gripper jaws; and
a centrifuge, in which a motor drives a support frame, and at least one rotary tube is suspended from the support frame; the second robotic arm uses the gripper jaws to grip the harvest bottle containing cell culture liquid and controls the placement of the harvest bottle into the rotary tube; the motor drives the support frame to rotate, and causes the rotary tube to swing outward, using centrifugal force to deposit the cells into the harvest tube.

2. The harvest system for an automated cell culture room according to claim 1, wherein the arm top section is connected to a first linear guide rail, and the first robotic arm controls the first linear guide rail to be normally oriented vertically relative to the ground, with a first slider block and a second slider block slidably disposed on the first linear guide rail, the lower gripper arm being coupled to the first slider block, and the upper gripper arm being coupled to the second slider block.

3. The harvest system for an automated cell culture room according to claim 1, wherein the harvest tube is formed with a valve chamber, and a valve body is disposed within the valve chamber, the valve body being penetrated by a flow channel, and rotation of the valve body allows the flow channel to either communicate with or seal off the interior of the harvest tube.

4. The harvest system for an automated cell culture room according to claim 1, wherein the outer surface of the bottle body is formed with a first collar, and the bottle body is secured to the rotary tube of the centrifuge by the first collar; the outer surface of the bottle body is further formed with a second collar, and the region between the first collar and the second collar is configured to allow the gripper jaw of the second robotic arm to grip and transfer the bottle body.

5. The harvest system for an automated cell culture room according to claim 1, wherein the lid assembly is provided with a window, and a coupling section protrudes adjacent to the window; the coupling section is pivotally connected to a cover plate via a latch pin, and the latch pin is further fitted with a torsion spring, the torsion spring being elastically compressed between the cover plate and the coupling section to normally bias the cover plate into a closed position covering the window.

6. The harvest system for an automated cell culture room according to claim 1, wherein the transport cart is provided with at least one insertion slot, and the pipette tube is positioned against the insertion slot such that the tube body is naturally oriented downward; the transport cart further extends downward to form a liquid collection tray, and the liquid collection tray is located directly beneath the tube body.

7. The harvest system for an automated cell culture room according to claim 1, wherein the centrifuge includes a housing, and the motor, support frame, and rotary tube are all housed within the interior of the housing; an operation window is formed at an upper portion of the housing, and the second robotic arm accesses the harvest bottle through the operation window; furthermore, a plurality of UV sterilization lamps are installed inside the housing to perform ultraviolet sterilization when the centrifuge is not in operation.

8. The harvest system for an automated cell culture room according to claim 1, wherein the gripper arm is connected to a second linear guide rail, and the second robotic arm controls the second linear guide rail to be normally oriented vertically with respect to the ground, wherein a third slider block is slidably disposed on the second linear guide rail, and the gripper jaw is coupled to the third slider block.

9. The harvest system for an automated cell culture room according to claim 1, wherein the harvest flask supply mechanism further comprises a harvest rail, and a plurality of harvest carts slidably disposed on the harvest rail, wherein the harvest bottle, after cell separation in the centrifuge, is retrieved by the second robotic arm and placed onto the harvest cart after the centrifugation operation is completed.

10. The harvest system for an automated cell culture room according to claim 1, wherein the pipette tube transport mechanism further comprises a recovery track, and a plurality of recovery carts slidably disposed on the recovery track, wherein the used pipette tube is placed onto the recovery cart by the first robotic arm after use.
